(19) United States
(12) Patent Application Publication
(10) Pub. No.: US 2005/0227906 A1
(43) Pub. Date: Oct. 13, 2005

(54) Fragrance compositions

(75) Inventors: Markus Schudel, Zurich (CH); Christian Ouellet, Bienne (CH); Marc Taschi, Zofingen (CH); Johnny Boumeesters, Zurich (CH)

Correspondence Address: NORRIS, MCLAUGHLIN & MARCUS, 875 THIRD AVE, 18TH FLOOR, NEW YORK, NY, 10022, US

(73) Assignee: GIVAUDAN SA, VERNIER (CH)

(21) Appl. No.: 10/507,039

(22) PCT Filed: Jan. 28, 2003
(86) PCT No.: PCT/CH03/00066

§ 371 Date: Apr. 4, 2005

(30) Foreign Application Priority Data
Mar. 27, 2002 (EP) 02006960.5

(51) Int. Cl. A61K 7/46

(52) U.S. Cl. 512/2

(57) ABSTRACT

The invention relates to microencapsulated fragrance compositions formed by spray drying an oil-in-water emulsion wherein the encapsulating material is a polymeric surfactant, for example polyvinyl alcohol. The encapsulating material contains no additional surfactant material.

This invention is concerned with fragrance compositions intended for use in perfumed articles and devices, and in particular compositions that can control the activation and diffusion of perfume in time, when exposed to moisture.

The prior art discloses microcapsules wherein a fragrance composition is encapsulated in a matrix material which protects the fragrance composition from its immediate environment and acts as means for the controlled diffusion of fragrance.

A particular example of such a composition is disclosed in U.S. Pat. No. 4,803,195 (Firmenich). There is disclosed a perfuming composition with deodorant or antiperspirant action which controls the activation and diffusion of fragrance over time when exposed to moisture, in particular sweat. The microcapsules are formed of a solid film-forming polymer, for example modified starch or polyvinyl alcohol, and an emulsifying agent. The emulsifying agents are low molecular weight (Mw) materials that are chosen from octenyl succinte-substituted starches, mono- or di-glycerides of fatty acids, esters derived from fatty acids and sorbitol or a saccharide, or their alkoxylated derivatives, or an ester of tartaric, citric, ascorbic, or lactic acid. This document teaches that the particular combination of the film-forming polymer and the emulsifier permits of a process of “re-encapsulation”. This “re-encapsulation” phenomenon allows repeated activations of perfume without the need of multiple applications of composition to the skin. Activation occurs upon exposure of the composition to moisture. When the source of moisture is removed, the composition re-encapsulates the fragrance to avoid further release.

U.S. Pat. No. 5,508,259 also to Firmenich, discloses similar compositions. The compositions comprise film-forming polymer and emulsifier as hereinabove described. Similarly, it is the combination of film-forming polymer and emulsifying agent that permits of the so-called “re-encapsulation” phenomenon.

EP 480 520 and U.S. Pat. No. 5,733,272 disclose encapsulated fragrance compositions. The former document uses as the encapsulating material the emulsifiers N-Lok or Purity Gum BE, which are both octenyl succinate-modified starches and available from National Starch and Chemical Company. The latter document discloses as encapsulating materials polysaccharides, such as modified starches.

A problem with these prior art compositions is that they use conventional emulsifying agents. Conventional emulsifying agents are all relatively low molecular weight materials, that is, they have molecular weights M_{w} considerably below 10,000 Daltons. These low molecular weight materials can desorb quite rapidly from the oil-water interface and may cause reversible or irreversible destabilisation of the dispersed oil phase, for example through coalescence, phase inversion, Ostwald ripening, flocculation, creaming or sedimentation. Further, whereas these prior art compositions activate to release fragrance before re-encapsulating under dry conditions, compositions containing low molecular weight emulsifying agents may be somewhat unstable in the presence of continuously moist or humid conditions, and may not resist rapid loss of large amounts of fragrance material.

Yet another problem with these formulations is that certain of the disclosed materials, such as polysaccharides, in particular modified starches, more particularly octenyl succinate-substituted starches tend to discolour, e.g. to take on a greyish or brownish hue, when exposed to high relative humidity or ambient product or body moisture. Naturally, this is unpleasant for end users.

DE 1909861 A, U.S. Pat. No. 3,772,215A and U.S. Pat. No. 4,906,488 describe fragrance delivery systems wherein a fragrance is incorporated into a water-sensitive, e.g. water-swellable or water-soluble, matrix. In these systems the fragrance is essentially dissolved molecularly in the polymer matrix to form a single phase, rather than the fragrance being held in discrete dispersed phases within a matrix material. The delivery system can then be incorporated into a suitable consumer product base. A problem with such monolithic systems is that upon exposure to moisture the matrix material simply dissolves to release the fragrance in an uncontrolled manner. This can be a particular problem in many bases that contain free-water within their structures, in which case the fragrance is free to diffuse continuously out of the product.

Such a system (predicated on the dissolution of the polymer matrix to release perfume) is completely unsuited for applications that are intended to give multiple releases of fragrance as the system is periodically or sequentially exposed to sources of ambient moisture.

U.S. Pat. No. 4,908,233A discloses a microencapsulated fragrance composition. Characteristic of microcapsules formed by a coacervation process, they consist of a fragrance oil reservoir surrounded by a very thin polymeric membrane. Such systems are often referred to in the art as “reservoir” devices. They are generally accepted to be mechanically very unstable, and in fact, this property is often exploited in their use in certain flavour or fragrance delivery applications, wherein the delivery is intended to be initiated by mechanical force.

WO 94/19449 discloses a fragrance carrier system that consists of a mixture of a solid inorganic carrier material onto which a poorly water-soluble components of a fragrance are absorbed, and a water-soluble or water-sensitive matrix material that encapsulates the more water-soluble components fragrance. Exemplary of the inorganic fillers is silicon dioxide. In such as system, the fragrance is contained in an open-pored carrier which uncontrollably allows fragrance to evaporate once the matrix material dissolves. Furthermore, certain fragrance ingredients may be unstable in the presence of inorganic carriers such as silcon dioxide. Still further, there are certain hazards associated with the use of silicon dioxide that may make for more complicated formulation and storage of products containing same, and may even adversely impact on consumer acceptance.

Rather similar to the disolved fragrance systems described above, this carrier system also releases its entire fragrance load from the open-pored carrier once contacted with moisture, and as such it too is completely unsuited for applications that are intended to give multiple releases of fragrance as the system is periodically or sequentially exposed to sources of ambient moisture.

### SUMMARY OF THE INVENTION

The present invention is directed to a fragrance carrier system that is adapted to release fragrance in a controlled manner when exposed to sources of ambient moisture such as sweat, but which effectively suppresses fragrance release when exposed to low humidity or dry environments. The system is also adapted to give multiple and sequential release of fragrance when exposed to cycles of moist and dry conditions.

As will be described in greater detail below, we have found that the use of fragrance dispersed in continuous matrix-type devices, and the judicious selection of the matrix material, enables the development a fragrance carrier system that is eminently suited to deliver fragrance in the manner described in the preceeding paragraph, and is not afflicted by any of the disadvantages of similar prior art devices.

Therefore, the present invention in a first aspect relates to a fragrance composition in multi-particulate form wherein the particles consist of a continuous phase of a polymeric surfactant material, and a plurality of dispersed phases consisting of fragrance containing oil droplets.

Polymeric surfactants (or macromolecular surfactants) are defined in more detail below. However, in general they are defined as surfactants that are capable of forming structured interfacial films around the dispersed phases. Furthermore, by virtue of their having high molecular weights, e.g. in the order of 10,000 daltons or more, they provide a steric barrier towards coalescence of adjacent dispersed phases, and thereby stabilise the dispersed phases. As their bulk renders them relatively immobile, whereas they swell in the presence of water to permit egress of fragrance, they retain their structural integrity at the dispersed phase interface even after prolonged exposure to water. This is in contradistinction to coventional low molecular weight emulsifiers that tend to desorb rapidly from the surface of the oil droplet interface, thereby compromising the interfacial film integrity and adversely affecting the the film's ability to “re-encapsulate”. Fragrance formulations formed using these emulsifiers have a relatively short-lived useful life as a consequence.

Furthermore, the use of polymeric surfactants precludes the need to employ conventional emulsifiers such as have been described above in relation to the prior art, and in particular it is not necessary to use polysaccharides, more particularly modified starches such as octenyl succinate-substituted starches. Avoidance of such materials has the added benefit that one can avoid the attendant disadvantages associated with the use of the materials.

Accordingly, in an specific embodiment of the invention, there is provided a composition as hereinabove defined preferably absent or substantially absent any polysaccharide emulsifying agents, in particular any modified starch emulsifying agents, more particularly any octenyl-succinate modified starch emulsifying agents.

The present invention also relates to articles of manufacture containing the aforesaid fragrance.

A method of manufacture of the fragrance composition forms yet another aspect of the invention. Fragrance compositions are prepared from emulsions containing a dispersed oil phase containing a fragrance material, and an aqueous, polymeric surfactant-containing continuous phase, the particles being formed when the emulsion is dried using techniques well known in the art, for example spray-drying.

Multiparticulate forms produced from emulsions offer the most effective way of ensuring that a very fine dispersed phase consisting of fragrance containing oil droplets. A very finely dispersed phase is an effective way of increasing the stability of the oil droplets in a matrix, and of reducing the incidence and amount of surface oil, by which is meant, in this manner very little fragrance-containing oil is expressed on the surface of the particles as a result of their manufacture.

### DETAILED DESCRIPTION OF THE INVENTION

Polymeric surfactants useful in the present invention may be selected from those water-soluble or water-swellable synthetic polymers known in the art as polymeric emulsifiers or colloid-stabilising polymers.

Examples of suitable polymeric surfactants are:
(1) Homopolymers, e.g.
   polyvinyl compounds, e.g. poly(vinyl acetate), poly(vinyl alcohol) or poly(vinyl pyrrolidon);
   polycarboxylic acids, e.g. poly(acrylic acid) and poly(methacrylic acid);
   polysulfonic acids, e.g., poly(styrene sulfonic acid);
   polyesters, e.g. glycol polyacrylate;
   polyamides, e.g. poly(acryl amide);
   polyurethanes, such as polyurethane that contains ionic groups, e.g. carboxylic acid, sulfonic acid or tertiary amines; or such as polyurethane that contains nonionic hydrophilic groups, e.g. ethylene oxide;
   poly(ethylene oxide), poly(propylene oxide) and further polyalkylene glycol derivatives;
(2) polycondensates, for example
   ethoxylated phenol-formaldehyde resins
   sulfonated aromatic formaldehyde resins
   urea or melamine-formaldehyde compounds
   polyamide, polyamine, and epichlorohydrin resins
(3) AB copolymers (wherein A is a more water-soluble or water swellable moiety and B is a less water-soluble or water-swellable moiety) selected from
   styrene copolymers, e.g. styrene-acrylic acid polymers or styrene-ethylene oxide polymers
   copolymers of polyvinyl and maleic acid compounds, e.g. styrene-maleic anhydride polymers or vinyl acetate-maleic acid ester polymers
   polyvinyl-polyalkylene copolymers, e.g. vinyl acetate-ethylene polymers, ethylene-acrylic acid-acrylic acid ester polymers or ethylene-acrylic acid-acrylonitrile polymers
   other vinyl copolymers, e.g. vinyl acetate polymers, acrylic acid-acrylonitrile polymers, acrylic acid-acryl amide polymers;
(4) ABA block copolymers selected from
   for “A” water-soluble or water-swellable moieties such as poly(ethylene oxide), poly(vinyl alcohol), poly(acrylic amid), poly(acrylic acid), poly(vinyl pyrrolidon), or poly(caprolactone);
   for “B” less water-soluble or sparingly water-soluble moieties such as poly(propylene oxide), poly(vinyl acetate), poly(vinyl butyral), poly(lauryl methacrylate), polystyrene, poly(hydroxystearic acid), polysiloxane,
(5) B(A)ₙ graft or comb polymers selected from
   for “A” from water-soluble or water-swellable moieties such as vinyl alcohol, vinyl acetate, ethylene oxide, propylene oxide, vinyl sulphonates, acrylic acids and vinyl amines,
   “B” is selected from vinyl polymer chains or siloxane chains,
(6) Natural polymeric emulsifiers such as cellulose derivatives, e.g. carboxymethylcellulose, hydroxypropylmethylcellulose, methylcellulose and other derivatives thereof,

In addition, plasticisers may be employed to modify the manner in which the polymeric surfactant releases fragrance material. Suitable plasticisers such as triacetin, triethyl citrate, polyethylene glycol, diethyl phthalate, tributyl citrate, glycerine or other conventional plasticizers and their mixtures.

Polymeric surfactants are of high molecular weight, in particular they are materials having molecular weights M_{w} of 10000 Daltons or more.

In a preferred embodiment the polymeric surfactant is a polyvinyl alcohol, for example those selected from commercially available products such as Mowiol® (ex Clariant SA, Switzerland). In particular, those PVAs having a degree of hydrolysis of between 70 and 100%, more particularly between 80 and 95%, with the highest emulsifying power being met at 88% hydrolysis.

Preferred polyvinyl alcohols have a viscosity measured at room temperature and as a 30% (wt) solution in water at a shear rate of 100 s⁻¹ lower than about 10,000 mPas, more preferably below about 5,000 mPas. Polymers having viscosities above this level become increasingly difficult to use in standard commercially available spray driers, granulators or other manufacturing equipment using nozzle-feeding devices. In order to ensure that the polymer has viscosity in the desired range, one preferably employs a polyvinyl alcohol having a molecular weight M_{w} of above 10,000 Daltons but lower than about 40,000 Daltons.

In a preferred embodiment the viscosity of polyvinyl alcohol is further reduced by oxidative breakdown of the polymer chains. In particular, residual 1,2-glycol units present in the native polymer are cleaved oxidatively. This process may be carried out under mild conditions in a manner known per se, for example using sodium periodate at a level of about 1 to 5% (wt) in a 20 to 30% solution in water at room temperature. The 1,2-glycol cleavage with sodium periodate takes place virtually spontaneously even at room temperature. Breakdown of the polymer chains with other oxidising agents, e.g. peroxides could also be used but mostly under harsher conditions. The quantities of peroxide needed to achieve a given viscosity and the reaction conditions have to be determined individually for each grade of polyvinyl alcohol as further described in the publically available literature for Mowiol®, (Clariant AG, Switzerland).

The polymeric surfactant may be present in amounts of 20 to 99% (wt) of the total fragrance composition, and it may be used individually or as mixtures of two or more polymeric surfactants.

Fragrance materials for use in compositions of the present invention may be selected from natural products such as essential oils, absolutes, resinoids, resins, concretes, and synthetic perfume components such as hydrocarbons, alcohols, aldehydes, ketones, ethers, acids, acetals, ketals and nitriles, including saturated and unsaturated compounds, aliphatic, carbocyclic and heterocyclic compounds, or precursors of any of the above. Other examples of odorant compositions which may be used are described in H 1468 (United States Statutory Invention Registration).

The amount of perfume possible to be incorporated into the fragrance composition may be up to 50 wt %, in some cases even up to 80 wt %, e.g. 1 to 80% based on dry material, with a high perfume retention close to 100% of even the very volatile components having a Loss Factor of greater than 10² Pa ppm. The term <<Loss Factor>> refers to a parameter that is related to the losses of fragrance material during drying and is defined as the product of the pure component vapour pressure (Pa) and the water solubility (ppm) at room temperature (see for example EP 1 208 754). Vapour pressures and water solubility data for commercially available fragrance components are well known and so the Loss Factor for a given fragrance component may be easily calculated. Alternatively, vapour pressure and water solubility measurements may be easily taken using techniques well known in the art. Vapour pressure of fragrance components may be measured using any of the known quantitative headspace analysis techniques, see for example Mueller and Lamparsky in Perfumes: Art, Science and Technology, Chapter 6 “The Measurement of Odors” at pages 176-179 (Elsevier 1991). The water solubility of fragrances may be measured according to techniques known in the art for the measurement of sparingly water-soluble materials. A preferred technique involves the formation of a saturated solution of a fragrance component in water. A tube with a dialysed membrane is placed in the solution such that after equilibration an idealised solution is formed within the tube. The tube may be removed and the water solution therein extracted with a suitable organic solvent to remove the fragrance component. Finally the extracted fragrance component may be concentrated and measured, for example using gas chromatography. Other methods of measuring fragrances are disclosed in Gygax et al, Chimia 55 (2001) 401-405.

Compositions according to the present invention may contain other optional excipients, for example anti-static agents, flowing agents, and other agents commonly known in the art. Excipients may be present in amounts of up to about 1% (wt) of the total composition.

The fragrance compositions of the present invention may be provided in multi-particulate form, for example as a spray-dried powder. Preferably, the particle size is between 0.01 and 2 mm. However, further processing of compositions is contemplated. For example, the compositions may be granulated in order to increase the particle size of the formulation which may be required for certain applications, e.g. when the formulation is to be mixed with a coarse material. Alternatively, the composition of the present invention may be dispersed in a matrix material, e.g. a hydrophilic glassy matrix material according to an extrusion process.

Fragrance compositions according to the present invention are suited for incorporation into consumer products, for example personal and household care products, and in particular said products which, in use, come into contact with moisture, e.g. deodorants, soaps and detergents, or diapers, humidity absorbers, air-fresheners and related air-purification products.

In a particularly preferred embodiment the fragrance composition may be admixed with a humidity adsorbing agent such as CaCl₂ and the like, in an air purification and room deodorising device as well as for the use in households where ever moisture occurs accompanied by malodour, e.g. in closets, wardrobes, items of apparel such as shoes, and waste bins.

The amount of fragrance composition employed in perfumed products or articles according to the present invention may vary according to the particular application in which it is employed and on the fragrance loading in the fragrance composition, but may typically be chosen from 0.05 to 20% based on the weight of the perfumed product or article.

Fragrance compositions of the present invention are preferably formed by drying an emulsion consisting of a fragrance-containing dispersed oil phase, and a continuous phase consisting essentially of water and a polymeric surfactant matrix material as hereinabove defined.

The drying step is preferably carried out in a manner known per se. For example, the emulsion may be spray-dried, spray-chilled, granulated, agglomerated or extruded according to methods known per se. Preferred manufacturing methods are disclosed in the Examples.

The skilled person will appreciate that the drying process need not eliminate all of the water from the system, and in fact some residual moisture will be retained in the “dried” particles. In order to ensure relatively quick processing times and thereby to ensure high encapsulation efficiency and good retention of fragrance oil, it is preferable that the amount of water to be eliminated by the drying is in the order of lower than 80%.

By employing an emulsion system it is possible to form extremely fine fragrance containing droplets surrounded by the polymeric surfactant. In this manner, it is possible to achieve very high fragrance loading and very low surface oil.

The drying step removes water to provide the fragrance composition in particulate form. The particles consist of a continuous matrix of polymeric surfactant that is mechanically very robust, and is very effective in preventing the coalescence of the oil droplets.

Preferably, at least 1% by weight, and preferably up to 80% by weight of the fragrance composition is formed by the liquid fragrance oil droplets. The mass fraction of liquid fragrance oil to solid polymer matrix may be measured by standard pulsed NMR techniques, or magnetic imaging techniques as further described in E. Dickinson, Advances In Food Colloids, Blackie Academic and Professional, 1996 at pages 145 through 175. As such, this is a very powerful technique to determine whether the correct phase-separated dipersed phases and matrix phase characteristic of particles of the present invention have been achieved.

There now follows a series of Examples that serve to illustrate embodiments of the present invention.

### EXAMPLE 1

Preparation of Encapsulates According to the Invention:

10.0 kg of polyvinyl alcohol Mowiol® 4-88, (Clariant AG, Switzerland) were dissolved in 90.0 kg deionised hot water (70° C.) to result in 10% polyvinyl alcohol solution. After cooling down to ambient room temperature 11.3 kg of a typical air freshener perfume (Givaudan Vernier SA, Switzerland) were added and homogenised using a Ultra-Turrax T-52 (IKA GmbH, Germany) at maximum speed for 5 minutes. The resulting emulsion had a water content of ca 80 wt % and a perfume droplet size of 0.8 μm measured with Olympus BX50 light microscope (Olympus, Japan). The dynamic viscosity of the emulsion was 47 mPa·s (shear rate: 100 s⁻¹) measured with a Modular Compact Rheometer MCR 300 (Physica, Germany).

This emulsion was further spray dried using standard spray-drying units, preferably having a water evaporation capacity of 33 kg/h (air throughput of 1,500 m³/h) at 150° C. inlet and 70° C. outlet temperature and further equipped with a two fluid nozzle operated at 3.0 bar air pressure.

The resultant powder had a total oil content of 51.2 wt % (0.6 wt % surface oil content) measured by standard HPLC procedures compared to a theoretical payload of 53.0 wt % perfume resulting in a perfume oil recovery (initial retention) of 97% related to the HPLC total oil measurements. The total oil content measured by pulsed NMR method using an Oxford MQA6005 (Oxford Instruments IAG, UK) resulted in 53 wt % total oil content (corrected for a residual moisture content of 3 wt % measured by Karl-Fischer) and reflecting that close to 100 wt % of the perfume is present as liquid droplets dispersed in a polymer matrix.

### EXAMPLE 2

Preparation of a Comparative Sample (Sample 1):

The sample 1 was prepared by a similar procedure to that described above for the sample of the present invention whereby the matrix material and the emulsifier where dissolved in water prior to homogenising it with the same air freshener perfume used for the sample of the present invention. Furthermore, for Sample 1 a Niro Mobile Minor (Niro A/S, Denmark) was used with a rotary atomiser and an inlet temperature 170° C. and an outlet temperature 80 . . . 84° C.

TABLE 1
Preparation of Sample 1 encapsulate

| | Matrix Material | Emulsifier | | |
|---|---|---|---|---|
| | maltodextrin DE = 6 | Capsul ® | Perfume | Water |
| Sample | [g] | [g] | [g] | [g] |
| 1 | 693 | 189 | 490 | 1280 |

Capsul ®: mod. starch emulsifier (National Starch and Chemicals Ltd., UK)
Perfume: same air freshener perfume as used above
Sample 1 based on U.S. Pat. No. 4,803,195 (Firmenich)

TABLE 2
Properties of Sample 1 encapsulate.

| | | | Initial | Residual moisture |
|---|---|---|---|---|
| | Total oil NMR | Theor. Payload | Retention | content |
| Sample | [wt %] | [wt %] | [%] | [wt %] |
| 1 | 30 | 36 | 83 | 4 |

By way of comparison, the inventive composition of Example has a total oil of 53%; a Theoretical Payload of 53%; an Initial Retention of 97%; and a residual moisture content of 3%. These data clearly demonstrate that inventive compositions give a much higher payload, a much higher retention and much reduced surface oil.

### EXAMPLE 3

Application Tests:

a) Storage Test:

20.0 g of standard CaCl₂ granulates used for humidity absorber products containing the encapsulates (sample of the present invention and Sample 1) on a 0.4 wt % perfume level and a blank containing no perfume or encapsulates were placed in 100 ml bottles and 10.0 g of deionised H₂O were added. The open bottles were placed in a climate chamber for 3 days at 37° C. and 70% humidity. After 3 days the coloration were inspected visually (see table 3).

TABLE 3
Visual evaluation of storage test.

| | Total oil | | |
|---|---|---|---|
| Capsule | [wt %] | Coloration | Preferred |
| sample(invention) | 53 | white | 1^{st} |
| CaCl₂ blank | — | white | 1^{st} |
| Sample 1 | 30 | yellow-orange | worst |

b) Perfume Release Test:

20 g of CaCl₂ granulates mixed with encapsulates on a perfume level of 0.4 wt % were placed in an open box in a closed climate room of ca. 2 m³ at 25° C. and 60 . . . 70% relative humidity. After 7 days the air in the room were olfactory evaluated by experts whereas the following results were found given in table 4:

TABLE 4
Olfactory evaluation of application test.

| | Payload | | | |
|---|---|---|---|---|
| Capsule | [wt %] | Intensity | Quality | Preferred |
| sample | 53 | very strong | intact perfume | 1^{st} |
| (invention) | | | | |
| Sample 1 | 31 | strong | intact perfume | 2^{nd} |

In addition, the inventive sample was described by perfumers as “fresh” whereas the Sample 1 was referred to as “humid”.

1. A fragrance composition comprising particles of a dried emulsion, wherein said particles comprise a matrix of a polymeric surfactant material, having dispersed therein liquid droplets of fragrance-containing oil.

2. A fragrance composition according to claim 1 wherein the polymeric surfactant is selected from the group consisting of:
I) Homopolymers, selected from polyvinyl compounds, such as poly(vinyl acetate), poly(vinyl alcohol) or poly(vinyl pyrrolidon); polycarboxylic acids, such as poly(acrylic acid) and poly(methacrylic acid); polysulfonic acids, such as poly(styrene sulfonic acid); polyesters, such as glycol polyacrylate; polyamides, such as poly(acryl amide); polyurethanes, such as polyurethane that contain ionic groups, such as carboxylic acid, sulfonic acid or tertiary amines; or a polyurethane that contains nonionic hydrophilic groups, such as ethylene oxide; poly(ethylene oxide), poly(propylene oxide) and polyalkylene glycol derivatives;
II) polycondensates, such as ethoxylated phenol, formaldehyde resins, sulfonated aromatic formaldehyde resins, urea or melamine, formaldehyde compounds, polyamide, polyamine, and epichlorohydrin resins;
III) AB copolymers wherein A is a more water-soluble or water swellable moiety and B is a less water-soluble or water-swellable moiety selected from styrene copolymers, such as styrene-acrylic acid polymers or styrene-ethylene oxide polymers, copolymers of polyvinyl and maleic acid compounds, such as styrene, maleic anhydride polymers or vinyl acetate, maleic acid ester polymers, polyvinyl-polyalkylene copolymers, such as vinyl acetate-ethylene polymers, ethylene-acrylic acid-acrylic acid ester polymers or ethylene-acrylic acid-acrylonitrile polymers, vinyl copolymers, such as vinyl acetate polymers, acrylic acid-acrylonitrile polymers, acrylic acid-acryl amide polymers;
IV) ABA block copolymers selected from polymers wherein for “A” water-soluble or water-swellable moieties such as poly(ethylene oxide), poly(vinyl alcohol), poly(acrylic amid), poly(acrylic acid), poly(vinyl pyrrolidon), or poly(caprolactone); and for “B” less water-soluble or sparingly water-soluble moieties such as poly(propylene oxide), poly(vinyl acetate), poly(vinyl butyral), poly(lauryl methacrylate), polystyrene, poly(hydroxystearic acid), polysiloxane,
V) B(A)n graft or comb polymers selected from for “A” from water-soluble or water-swellable moieties such as vinyl alcohol, vinyl acetate, ethylene oxide, propylene oxide, vinyl sulphonates, acrylic acids and vinyl amines, “B” is selected from vinyl polymer chains or siloxane chains,
VI) Natural polymeric emulsifiers such as cellulose derivatives, such as carboxymethylcellulose, hydroxypropylmethylcellulose, methylcellulose and other derivatives thereof.

3. A fragrance composition according to claim 1 wherein the polymeric surfactant is polyvinyl alcohol having a molecular weight of more than 10,000 Daltons and less than 40,000 Daltons.

4. A fragrance composition according to claim 3 wherein the polyvinyl alcohol has a degree of hydrolysis of between 70% and 100%.

5. A fragrance composition according to claim 1 wherein polysaccharide emulsifying agents are excluded from the fragrance compositions.

6. A fragrance composition according to claim 1 wherein modified-starch emulsifying agents are excluded from the fragrance compositions.

7. A fragrance composition according to claim 1 wherein octenyl succinate-modified starch emulsifying agents are excluded from the fragrance compositions.

8. A perfumed article containing a fragrance composition according to claim 1.

9. A perfumed article according to claim 8 wherein said perfumed article is a deodorant composition.

10. A perfumed article according to claim 8 wherein said perfumed article is a humidity absorber.

11. A perfumed article according to claim 10 comprising calcium chloride.

12. A perfumed article according to claim 8 wherein the perfumed article is a soap.

13. A process of encapsulating a fragrance material comprising the steps of:
dispersing or emulsifying an oil phase containing fragrance material in a continuous phase consisting essentially of water and a polymeric surfactant according to claim 1, and
thereafter spray drying the emulsion to remove water.

14. A fragrance composition consisting of particles of a dried emulsion, wherein said particles comprise a matrix of a polymeric surfactant material, having dispersed therein liquid droplets of fragrance-containing oil.

15. A fragrance composition consisting of particles of a dried emulsion, wherein said particles consist of a matrix of a polymeric surfactant material, having dispersed therein liquid droplets of fragrance-containing oil.
